(19) United States
(12) Patent Application Publication
(10) Pub. No.: US 2004/0043384 A1
(43) Pub. Date: Mar. 4, 2004

(54) In vitro protein translation microarray device

(76) Inventor: Andrew V. Oleinikov, Mill Creek, WA (US)

Correspondence Address: Jeffrey B. Oster, Combimatrix Corporation, 6500 Harbour Heights Parkway, Mukilteo, WA, 98275, US

(21) Appl. No.: 10/229,714

(22) Filed: Aug. 28, 2002

(51) Int. Cl. C12Q 1/68; C12M 1/34; C12P 21/06

(52) U.S. Cl. 435/6; 435/287.2; 435/69.1

(57) ABSTRACT

There is disclosed a multiple format protein microarray, and a process for synthesizing the multiple format protein mircoarray.

### CROSS-REFERENCE TO RELATED APPLICATION

This patent application claims priority from U.S. provisional patent application No. 60/315,253 filed Aug. 27, 2001.

### TECHNICAL FIELD OF THE INVENTION

The present invention provides a plurality of proteins spatially arranged on a microarray device and a process for synthesizing the protein microarray.

### BACKGROUND OF THE INVENTION

In the world of microarrays, biological molecules (e.g., oligonucleotides, polypeptides and the like) are placed onto surfaces at defined locations for potential binding with target samples of nucleotides or receptors. Microarrays are miniaturized arrays of biomolecules available or being developed on a variety of platforms. Much of the initial focus for these microarrays have been in genomics with an emphasis of single nucleotide polymorphisms (SNPs) and genomic DNA detection/validation, functional genomics and proteomics (Wilgenbus and Lichter, J. Mol. Med. 77:761, 1999; Ashfari et al., Cancer Res. 59:4759, 1999; Kurian et al., J. Pathol. 187:267, 1999; Hacia, Nature Genetics 21 suppl.:42, 1999; Hacia et al., Mol. Psychiatry 3:483, 1998; and Johnson, Curr. Biol. 26:R171, 1998).

There are, in general, three categories of microarrays (also called “biochips” and “DNA Arrays” and “Gene Chips” but this descriptive name has been attempted to be a trademark) having oligonucleotide content. Most often, the oligonucleotide microarrays have a solid surface, usually silicon-based and most often a glass microscopic slide. Oligonucleotide microarrays are often made by different techniques, including (1) “spotting” by depositing single nucleotides for in situ synthesis or completed oligonucleotides by physical means (ink jet printing and the like), (2) photolithographic techniques for in situ oligonucleotide synthesis (see, for example, Fodor U.S. Pat. No. 5,445,934 and the additional patents that claim priority from this priority document), (3) electrochemical in situ synthesis based upon pH based removal of blocking chemical functional groups (see, for example, Montgomery U.S. Pat. No. 6,092,302 the disclosure of which is incorporated by reference herein and Southern U.S. Pat. No. 5,667,667), and (4) electric field attraction/repulsion of fully-formed oligonucleotides (see, for example, Hollis et al., U.S. Pat. No. 5,653,939 and its duplicate Heller U.S. Pat. No. 5,929,208). Only the first three basic techniques can form oligonucleotides in situ that are, building each oligonucleotide, nucleotide-by-nucleotide, on the microarray surface without placing or attracting fully formed oligonucleotides.

With regard to placing fully formed oligonucleotides at specific locations, various micro-spotting techniques using computer-controlled plotters or even ink-jet printers have been developed to spot oligonucleotides at defined locations. One techniques loads glass fibers having multiple capillaries drilled through then with different oligonucleotides loaded into each capillary tube. Microarray chips, often simply glass microscope slides, are then stamped out much like a rubber stamp on each sheet of paper of glass slide. It is also possible to use “spotting” techniques to build oligonucleotides in situ. Essentially, this involves “spotting” relevant single nucleotides at the exact location or region on a slide (preferably a glass slide) where a particular sequence of oligonucleotide is to be built. Therefore, irrespective of whether or not fully formed oligonucleotides or single nucleotides are added for in situ synthesis, spotting techniques involve the precise placement of materials at specific sites or regions using automated techniques.

Another technique involves a photolithography process involving photomasks to build oligonucleotides in situ, base-by-base, by providing a series of precise photomasks coordinated with single nucleotide bases having light-cleavable blocking groups. This technique is described in Fodor et al., U.S. Pat. No. 5,445,934 and its various progeny patents. Essentially, this technique provides for “solid-phase chemistry, photolabile protecting groups, and photolithography . . . to achieve light-directed spatially-addressable parallel chemical synthesis.” Binary masks are used in the preferred embodiment The electrochemistry platform (Montgomery U.S. Pat. No. 6,092,302, the disclosure of which is incorporated by reference herein) provides a microarray based upon a semiconductor chip platform having a plurality of microelectrodes. This microarray design uses Complimentary Metal Oxide Semiconductor (CMOS) technology to create high-density arrays of microelectrodes with parallel addressing for selecting and controlling individual microelectrodes within the array. The electrodes turned on with current flow generate electrochemical reagents (particularly acidic protons) to alter the pH in a small “virtual flask” region or volume adjacent to the electrode. The microarray is coated with a porous matrix for a reaction layer material. Thickness and porosity of the material is carefully controlled and biomolecules are synthesized within volumes of the porous matrix whose pH has been altered through controlled diffusion of protons generated electrochemically and whose diffusion is limited by diffusion coefficients and the buffering capacities of solutions. However, in order to function properly, the microarray using electrochemistry means for in situ synthesis has to alternate anodes and cathodes in the array in order to generated needed protons (acids) at the anodes so that the protons and other acidic electrochemically generated acidic reagents will cause an acid pH shift and remove a blocking group from a growing oligomer.

Protein Microarrays

Fast and efficient immobilization of different proteins on microarrays (protein microarray) will facilitate rapid diagnostics or identification of drug-lead by analyzing thousands of proteins in parallel for protein-target interactions and/or for catalytic or inhibitory effects of various enzymes. The goal is to prepare and control the three-dimensional patterning of these proteins on the microarray through nano-spotting or protein self-assembly. Previously described methods based on the use of spotting devices have a number of disadvantages and a limited flexibility in preparation of different custom arrays. Purification of thousands of proteins is also a laborious and expensive task.

Polypeptide microarrays have used the same procedures as have been used for oligonucleotide microarrays, that is, spotting, photolithography and electrochemical synthesis. However, with regard to in situ synthesis, it is much more expensive and difficult to make polypeptides of significant diversity than oligonucleotides in situ because of the simple fact that polypeptides are made from 20 naturally occurring L amino acids and oligonucleotides are made from only four different nucleotide bases. That cycling difference adds to the cost for in situ synthesis in an exponential manner.

The preparation and use of the protein microarrays is a significantly more difficult task than that of the gene (oligonucleotide) microrrays due to different chemical nature of the monomers composing the microarray. For example, nucleic acids with different sequences coding for different proteins are similar in their chemical features and have predictable binding partners. However, each protein is unique in its tertiary structure. Protein microarrays have been developed in several different formats each serving a special need. These formats include four major display systems: phage-display libraries, cell-based libraries, protein-mRNA/DNA libraries and non-biological microarray-based display (protein microarray) (reviewed in Li, Nat. Biotechnol. 18:1251-1256, 2000). Protein microarrays can be used for fast identification of protein-target interactions, catalytic or inhibitory effects of various enzymes, screening for proteins with desired features, and molecules that modify selected proteins in a desired way.

Several ways of manufacturing the protein microarrays have been described. The first approach is spotting or printing purified proteins on the surface of a microarray (MacBeath and Schreiber, Science 289:1760-1763, 2000; Zhu et al., Nat. Genet. 26:283-289, 2000). Usually the source proteins are purified from the tissues/cells or synthesized in vivo in bacterial or other system as recombinant molecules. The “spotting” approach has number of serious limitations. First, the spotting process is quite laborious and time-consuming, involving several steps for purification and/or recombinant technology manipulations and chemical modification of the proteins. Also, mammalian proteins that were made in recombinant bacterial systems are often not properly glycosylated, are often folded improperly, and are difficult to purify. Further, mammalian expression systems are inefficient, and the chemical modification in general may affect the protein integrity. Second, the “spotting” approach is not well suited for adjusting to the custom needs so that any change in microarray composition (protein selection or change of the position on the microarray) would require a number of laborious manipulations. Also, spotting of different proteins using currently available devices (such as pin spotters or ink-jet printers) often has a problem of cross-contamination due to necessity of cleaning the spotting parts of device). Finally, the protein spots will often dry during spotting since only nano-liter amounts usually applied to the microarrays (MacBeath and Schreiber, Science 289:1760-1763, 2000). Drying of the protein spots will affect the affinity of the spotted protein to properly bind with its target.

Another approach to construct protein microarrays is based on self-assembly of protein molecules conjugated to oligonucleotides (Brenner and Lerner, Proc. Natl. Acad. Sci. USA 89:5381-5383, 1992; and Niemeyer et al., Nucleic Acids Research 22:5530-5539, 1994) based upon conjugation of streptavidin (SA) to an oligonucleotide. This SA-oligonucleotide conjugate binds to the complementary oligonucleotide, providing specific addressing. The protein is labeled with biotin to provide placing of the protein to its specific address. The process involves simply immersing a membrane in a solution containing all of the proteins of interest. The immobilized proteins are exposed to a mild aqueous ambient environment to reduce the risk that the proteins will denature due to solvent evaporation, mechanical shearing or flash heating. The problem with this approach is a necessity to obtain purified proteins to be placed on the protein microarray and to label the purified proteins with biotin. A further problem with this approach and shared by any spotting or ink jet printing approaches is at the proteins must first be obtained and these are not trivial tasks, especially when many different proteins are desired on a single mircoarray device. For example proteins can be obtained by tissue or cell source extraction or synthesized in vivo using recombinant methods from bacterial or other expression systems. Either technique requires many purification steps and each purification process is often unique to the particular protein obtained. Thus, the step of obtaining a protein is highly labor intensive and not prone to automation when multiple different proteins are needed. In addition, there is a problem when mammalian proteins are synthesized in recombinant systems using prokaryotic cells as there are often problems with improper folding or improper glycosylation patterns.

Another approach of preparation of protein libraries (Nemoto et al., FEBS Lett 414:405-408, 1997; and Roberts and Szostak, Proc. Natl. Acad. Sci. USA 94:12297-12302, 1997) exploited the ability of the antibiotic puromycin to bind covalently to the nascent polypeptide during ribosomal translation of mRNA. Briefly, puromycin was covalently bound to the end of a mRNA molecule in which a stop-codon was deleted through its DNA oligonucleotide. When this construct was translated in vitro the ribosome stopped at the junction between mRNA and DNA and puromycin moiety had time to react with the nascent polypeptide. In the case of such reaction the nascent polypeptide was covalently bound to its own mRNA molecule. Unfortunately, this method was inefficient and several modifications had been made to improve the yield of mRNA-protein conjugates and to eliminate the necessity of stop-codon removal (Liu et al., Methods Enzymol. 318:268-293, 2000; and Kurz et al., Nucleic Acids Res. 28:E83, 2000). Protein microarrays using this approach have constructed based on the addressing of peptide-nucleic acid (NA) conjugates to the designed locations on the microarray device (WO 99/51773). However, the usability of this approach for this purpose generates serious doubts. The yield of peptide-NA conjugates is still too small for production of reasonable amounts of conjugates for their immobilization on a microarray device. This yield is limited not only by competition between ribosome dissociation without cross-linking and puromycin reaction but also, and mostly, by the fact that each mRNA can be read only once in this method. Therefore, there is a need to increase the translation of each mRNA to multiple times, but the puromycin approach does not allow for multiple copy translation with any modification. Therefore, to make the peptide-NA conjugates in amounts suitable for the microarray construction it would be necessary to use separate reactions for different mRNAs and compensate for the low yield of conjugates using enormous amounts of mRNA and cell-free lysate. This will create additional problems relevant to purification of conjugates and makes the overall process quite expensive.

Another approach of ribosome display (Mattheakis et al., Proc. Natl. Acad. Sci. USA 91:9022-6, 1994; Mattheakis et al., Methods Enzymol. 267:195-207, 1996; and Jermutus et al., Curr. Opin. Biotechnol. 9:534-548, 1998) keeps the nascent polypeptide and mRNA together through the ribosome, which is stalled at the end of an mRNA lacking a stop-codon. This approach has all the same pitfalls (plus additional problems of ribosome-mRNA complex instability) as cross-linked peptide-NA approach for the use it in protein microarray preparation.

Therefore, there is a need in the art to overcome the problem of multiple polypeptide diversity and increase yields and abundance at a site. Moreover, there is a need in the art to provide a diverse group of proteins on a mircoarray that have proper tertiary configurations so that any binding to such proteins on a protein mircoarray reflects a true protein-protein or protein-ligand interaction and not an artifact of a constrained protein configuration on a microarray device. The present invention addresses such needs with a new approach.

### SUMMARY OF THE INVENTION

The present invention provides a process for producing a self-assembled protein microarray having a plurality of proteins in discrete locations, comprising the steps of:

(a) preparing a plurality of cDNA's, each encoding a different protein, wherein each cDNA comprises a promoter region and a coding region and segregating each cDNA into separate chambers;

(b) transcribing each cDNA into a mRNA, wherein the mRNA will form a protein encoded by the coding region of the cDNA;

(c) translating each mRNA in a cell-free translation system to synthesize a plurality of synthetic proteins, wherein each synthetic protein includes a first binding moiety incorporated therein, and whereby each mRNA molecule can be used to translate a plurality of synthetic proteins to increase yield;

(d) attaching a second binding moiety that specifically binds to the first binding moiety, wherein the second binding moiety further comprises an oligonucleotide tag sequence to form a oligonucleotide-addressed synthetic protein; and

(e) localizing the oligonucleotide-addressed synthetic protein onto an oligonucleotide tag mircoarray device, wherein the oligonucleotide tag mircoarray device comprises a plurality of oligonucleotide sequences at known locations, wherein then oligonucleotide sequences are designed to be complementary to an oligonucleotide tag sequence on the second binding moiety, whereby each oligonucleotide-addressed protein localizes to its predefined complementary region on the oligonucleotide tag mircoarray device through nucleic acid hybridization.

Preferably, the promoter region of the plurality of cDNA is a promoter for RNA polymerase. Preferably, the cDNA's are prepared by nucleic acid amplification techniques, including PCR (polymerase chain reaction) techniques and TCR (transcriptase chain reaction) techniques. Preferably, the cDNAs further comprises a tag region that codes on expression for a protein tag, wherein the protein tag sequence is used to affinity bind the synthetic protein in order to wash out unbound first binding moiety. Preferably, the translating step (c) further comprises adding cell or liver microsomes in order to provide for eukaryotic cell glycosylation of the synthetic protein at N-linked or O-linked glycosylation sites. Preferably, the first binding moiety is biotin or a biotin derivative thereof, and the second binding moiety is streptavidin or a streptavidin derivative thereof, or the first binding moiety is an antigenic epitope and the second binding moiety is an antibody or fragment thereof that binds to the first moiety antigen. Most preferably, the first binding moiety is a biotin moiety that is linked to the synthetic polypeptide through Lys residues. Preferably, the oligonucleotide tag sequence attached to the second binding moiety is from about 12 to about 100 nucleotides in length wherein at least 12 nucleotides are exactly complimentary to their corresponding tag array oligonucleotide sequence on the mircoarray device.

The present invention provides a protein microarray having a plurality of proteins in discrete locations, wherein the protein mircoarray is produced by a process comprising the steps of:

(a) preparing a plurality of cDNA's, each encoding a different protein, wherein each cDNA comprises a promoter region and a coding region;

(b) transcribing each cDNA into a mRNA, wherein the mRNA will form a protein encoded by the coding region of the cDNA;

(c) translating each mRNA in a cell-free translation system to synthesize a plurality of different proteins, wherein each synthetic protein includes a first binding moiety incorporated therein, and whereby each mRNA molecule can be used to translate a plurality of synthetic proteins;

(d) attaching a second binding moiety that specifically binds to the first binding moiety, wherein the second binding moiety further comprises an oligonucleotide tag sequence to form a oligonucleotide-addressed synthetic protein; and

(e) localizing the oligonucleotide-addressed synthetic protein onto an oligonucleotide tag mircoarray device, wherein the oligonucleotide tag mircoarray device comprises a plurality of oligonucleotide sequences at known locations, wherein then oligonucleotide sequences are designed to be complementary to an oligonucleotide tag sequence on the second binding moiety, and whereby each oligonucleotide-addressed protein localizes to its predefined complementary region on the oligonucleotide tag mircoarray device through nucleic acid hybridization.

Preferably, the cDNA's are prepared through an amplification process including PCR (polymerase chain reaction) techniques and TCR (transcriptase chain reaction) techniques. Preferably, the cDNAs further comprises a tag region that codes on expression for a protein tag, wherein the protein tag sequence is used to affinity bind the synthetic protein in order to wash out unbound first binding moiety. Preferably, the translating step (c) further comprises adding cell or liver microsomes in order to provide for eukaryotic cell glycosylation of the synthetic protein at N-linked or O-linked glycosylation sites. Preferably, the first binding moiety is biotin or a biotin derivative thereof, and the second binding moiety is streptavidin or a streptavidin derivative thereof, or the first binding moiety is an antigen and the second binding moiety is an antibody of fragment thereof that binds to the first moiety antigen. Most preferably, the first binding moiety is a biotin moiety that is linked to the synthetic peptide through Lys residues. Preferably, the oligonucleotide tag sequence attached to the second binding moiety is from about 12 to about 100 nucleotides in length wherein at least 12 nucleotides are exactly complimentary to their corresponding tag array oligonucleotide sequence on the mircoarray device.

The present invention further provides an alternative process for producing a self-assembled protein microarray having a plurality of proteins in discrete locations, comprising the steps of: (a) preparing a plurality of cDNA's in separate containers, wherein each cDNA encodes a different protein; (b) amplifying each cDNA with specific primers to produce a plurality of synthetic proteins, wherein each synthetic protein contains a peptide tag at either terminus; (c) incorporating in each synthetic protein a first binding moiety; (d) capturing each synthetic protein on a solid phase using an antibody directed against the peptide tag; (e) adding a second binding moiety to each synthetic protein in each container, wherein the second binding moiety is conjugated to a plurality of different tag oligonucleotides and binds specifically to the first binding moiety, thereby forming an oligonucleotide-tagged protein complex, wherein the different tag oligonucleotides are designed in a way that they do not cross-hybridize to each other; (f) eluting each oligonucleotide-tagged protein complex from the solid phase; and (g) mixing the oligonucleotide-tagged protein complexes from separate containers and incubating the mixture with an oligonucleotide tag microarray device, wherein each oligonucleotide-tagged protein complex localizes to its predefined complementary region on the oligonucleotide tag microarray device, thereby forming a self-assembled protein microarray having a plurality of proteins in discrete locations.

Preferably, the cDNA's are prepared through PCR techniques. Still preferably, the first binding moiety is an antigenic epitope or a fragment thereof, and the second binding moiety is an antibody or a fragment thereof that binds to the first binding moiety. Or the first binding moiety is biotin or a biotin derivative thereof, and the second binding moiety is streptavidin or a strepavidin derivative thereof. The biotin moiety is linked to the synthetic protein through Lys residues. Still preferably, tag oligonucleotide conjugated to the second binding moiety is from about 10 to about 100 nucleotides in length, wherein at least 12 nucleotides are exactly complimentary to their corresponding oligonucleotide sequence on the oligonucleotide tag microarray device.

The present invention further provides a process for producing a self-assembled protein microarray having a plurality of proteins in discrete locations, comprising: (a) preparing a plurality of cDNA's in separate containers, wherein each cDNA encodes a different protein; (b) amplifying each cDNA with specific primers to produce a plurality of synthetic proteins, wherein each synthetic protein contains a peptide tag at either terminus; (c) incorporating in each synthetic protein a first binding moiety by in vitro translation; (d) capturing each synthetic protein on a solid phase using an antibody directed against the peptide tag; (e) adding a second binding moiety to each synthetic protein in each container, wherein the second binding moiety is multivalent and binds specifically to the first binding moiety; (f) adding a plurality of oligonucleotides labeled with the first binding moiety to bind to the second binding moiety, thereby forming an oligonucleotide-tagged protein complex, wherein the different tag oligonucleotides are designed in a way that they do not cross-hybridize to each other, (g) eluting each oligonucleotide-tagged protein complex from the solid phase; and (h) mixing the oligonucleotide-tagged protein complexes from separate containers and incubating the mixture with an oligonucleotide tag microarray device, wherein each oligonucleotide-tagged protein complex localizes to its predefined complementary region on the oligonucleotide tag microarray device, thereby forming a self-assembled protein microarray having a plurality of proteins in discrete locations.

Preferably, the cDNA's are prepared through PCR techniques. Still preferably, the first binding moiety is an antigenic epitope or a fragment thereof, and the second binding moiety is an antibody or a fragment thereof that binds to the first binding moiety. Or the first binding moiety is biotin or a biotin derivative thereof, and the second binding moiety is streptavidin or a strepavidin derivative thereof. The biotin moiety is linked to the synthetic protein through Lys residues. Still preferably, tag oligonucleotide conjugated to the second binding moiety is from about 10 to about 100 nucleotides in length, wherein at least 12 nucleotides are exactly complimentary to their corresponding oligonucleotide sequence on the oligonucleotide tag microarray device.

### BRIEF DESCRIPTION OF THE DRAWINGS

FIG. 1 shows a schematic of a protein array synthesis process wherein streptavidin-oligonucleotide conjugates serve both as capture moieties for biotin-labeled proteins and as addressing agent for self-assembling arrays. The proteins have a biotin moiety conjugated to a Lys residue, were introduced co-translationally, and were synthesized using a cell-free in vitro translation system with mRNA molecules from in vitro transcription using T7 RNA polymerase. The advantage of this process is a significantly greater yield of proteins, even in microgram per ml concentrations or quantities.

FIG. 2 shows a four-panel schematic for producing a protein microarray having non-constrained, tagged proteins that can be used for binding studies. In panel #1, a protein molecule is synthesized in vitro having biotinylated Lys residues and a peptide tag added to either the N terminus or the C terminus of the protein. Specifically, the sequence of the synthesized protein was controlled by the in vitro synthesis process, wherein natural mRNA or cDNA was amplified with specific primers to produce a recombinant molecule coding for the desired protein sequence and having a peptide tag at either end. The cDNA is transcribed and translated in vitro with the standard 20 amino acids except the Lys group added to protein using lysine-tRNA charged with biotinylated lysine. In panel #2, the tagged, synthesized protein is “captured” by an antibody specific for the tag (anti-tag antibody) wherein the anti-tag antibody is bound (at its fixed site) to a bead, resin or other hard surface. This capture of the protein allows for washing and removal of non-binding debris and un-incorporated biotinylated Lys. In panel #3, a streptavidin-oligonucleotide moiety (SA-oligo) is added to the immobilized protein and incubated to allow the SA moiety to naturally bind to biotin moieties. In panel #4, the complex is eluted from the immobilized solid phase by reaction condition to break the anti-tag/tag binding. The complex is now in solution and added to a microarray having oligonucleotide capture probes complementary to the oligonucleotide sequences in the SA-oligo moiety. Hybridization to specific sites creates an immobilized complex where the identity of the protein sequence is known and wherein the protein is available in solution for binding by tethered to the specific site on the protein microarray.

FIG. 3 shows a schematic for translation of a luciferase enzyme translated in vitro as a fusion protein with the addition of a FLAG eight amino acid additional sequence for immobilizing the translated fusion protein. A commercial anti-FLAG antibody (α-FLAG) was immobilized onto beads and was able to capture the translated fusion protein. Moreover, the ability of the translated protein to property fold (tertiary structure) was demonstrated in the insert showing light production catalyzed by the translated fusion protein.

FIG. 4 shows the results of a mircoarray image. SA was treated with Traut's reagent (Pierce) to introduce SH groups to the protein. Oligonucleotide 1 (oligo1) was synthesized with amino-group at the 3′ end. The modified protein was coupled to this oligo1 using heterobifunctional reagent maleimidobenzoyl-N-hydrosuccinimide ester (Pierce). This SA-oligo1 conjugate was incubated with biotin labeled with fluorescein-isotiocyanate (FITC) and then hybridized to the microarray device with 12 different oligonucleotides synthesized in specific order (oligo1 and oligos2-12 or “other 11 oligonucleotides”). The presence of biotin-FITC was detected using fluorescence microscope and CCD camera to provide the image shown.

FIG. 5 shows an approach wherein resin-bound luciferase-FLAG was incubated with SA-oligo1. The resin was washed and residual biotin binding sites on SA were blocked by incubation with biotin-FITC. The luciferase-FLAG:SA-oligo1 complexes were eluted from the resin by FLAG peptide and hybridized to the 12-oligos microarray as used in FIG. 4. Presence of luciferase on the microarray was demonstrated by incubation of the microarray with goat anti-luciferase antibody followed by incubation with anti-goat IgG antibody labeled with Cy5® fluorescent dye. The schema of this experiment is shown in the left panel and the data in the form of a CCD camera image is shown in the right panel. This experiment demonstrated that a protein with Mr of 60 kDa could be synthesized in cell-free system and successfully immobilized on an oligonucleotide tag array microarray device at desired address. After immobilization this protein preserved its ability to interact with other molecules that was demonstrated by its reactivity with antibody, a molecule with Mr of 150 kDa.

FIG. 6 shows four sites on a streptavidin (SA) tetramer that are stericly not accessible for interaction with other protein-incorporated biotin moieties when protein is bound to a solid support. The free sites can be filled with biotin-containing specific oligonucleotides to provide tag oligonucleotide sequences to localize specific synthetic proteins.

FIG. 7 shows an alternative scheme for protein microarray preparation wherein in vitro-translated proteins are attached with a peptide tag sequence. Specifically, natural mRNAs or cDNAs are amplified with specific primers to produce a recombinant molecule coding for a protein with a specific “tag” sequence at either the C terminus or N-terminus of the protein. Tag sequences include, for example, an eight amino acid FLAG sequence having a commercially available antibody to bind to it. The synthesized cDNA encoding the tagged protein are transcribed and translated in vitro with biotinylated lysine residues and in separate tubes or well of a microtiter plate.

FIG. 8 shows an illustration of an embodiment wherein the synthetic protein is first bound to a solid support after synthesis and molecules providing label (i.e., first binding moieties, biotinylated lysil-tRNA) are removed by washing. The synthesized and tagged protein is captured via the tag, such as through an anti-tag resin (i.e., a solid phase having a capture protein (e.g., antibody) specific to bind to the tag moiety). Other components of the mixture can be removed through washing.

FIG. 9 continues the process from FIG. 8 wherein an excess of a second binding moiety (e.g., streptavidin (SA)) is added to the resin-captured protein. This mixture is incubated to bind the SA. Excess second binding moiety is washed off. This step provides for the synthetic proteins to be bound to the second binding moieties to accelerate the process.

FIG. 10 shows the continuation of the process from FIG. 9 wherein any excess of specific oligonucleotides conjugated to the first binding moieties (e.g., biotinylated oligonucleotides) is added to bind and to tag the synthetic protein-second binding moiety (e.g., synthetic protein-SA shown) complex.

FIG. 11 continues the process from FIG. 10 wherein the FIG. 10 complex is eluted from the tag attached to a solid phase (bead or microarray) in less harsh conditions. The example of less harsh conditions shown is the addition of eluting peptide (to compete with the tag) as opposed to extreme pH conditions that would be too harsh for proper folding of the proteins.

FIG. 12 continues the process from FIG. 11 wherein the eluted protein complexes that have been eluted in FIG. 11 are mixed together.

FIG. 13 shows the mixture from FIG. 12 having the first binding moiety (i.e., oligonucleotide tag sequence) self-assembling onto a microarray device through complimentary binding (hybridization) of the oligonucleotide tag sequence first binding moiety of the protein complex with its corresponding sequence on the microarray device spotted or synthesized at known locations.

FIG. 14 shows an application of the inventive protein microarray for antibody-based detection of immobilized proteins having fluorescent tags to identify the locations of different proteins based upon antibody specificity.

FIG. 15 shows the preparation of a microarray device having two different proteins (Luciferase (Luc) and green fluorescent protein (GFP)) immobilized thereon as schematically shown in FIG. 14. Two different labels were attached to two antibodies to detect Luciferase with the label Texas Red® that fluoresced in the image in the upper panel and with the label Cy5® to detect GFP in the lower panel.

FIG. 16 shows the functional activity of GFP and Luc illustrated on an image of a protein microarray of the present invention. GFP was detected by fluorescence under appropriate blue light. Luc activity was detected using commercially available chemiluninescent substrate (Luciferase assay reagent, Promega) by generating light detected by a CCD camera.

### DETAILED DESCRIPTION OF THE INVENTION

The present invention provides a process for in vitro translation systems to produce properly folded proteins having native tertiary structure and active enzymatic activity (when appropriate) to be placed on the microarray. These in vitro synthesized proteins are located in known discrete regions on a microarray by adding self-addressing oligonucleotide tags that can self-assemble to a microarray having oligonucleotide content with capture probes designed to capture their corresponding proteins. The orientation of proteins self-assembled to a microarray but synthesized or translated in vitro provides for proper folding and less constraints from a microarray such that protein biological activity is preserved. Data provided herein show that this process works and an in vitro translated protein is specifically placed on the microarray and detected with corresponding antibody and by specific protein activity. This inventive method is capable of automation to provide microarray devices with theoretically unlimited numbers of different and separately functional proteins for binding, enzymatic activity or other biochemical interactions with a properly folded protein.

Current PCR technology allows for rapid assembly of any coding sequence that further contains upstream regulatory sequences to augment its transcription into the appropriate mRNA and can further contain a peptide tag sequence that can be accessed for post-translational manipulation and purification. In vitro protein biosynthesis allows for parallel design, synthesis and purification of thousands of different proteins that are properly folded and even allows for proper glycosylation where appropriate and necessary for activity (e.g., erythropoietin).

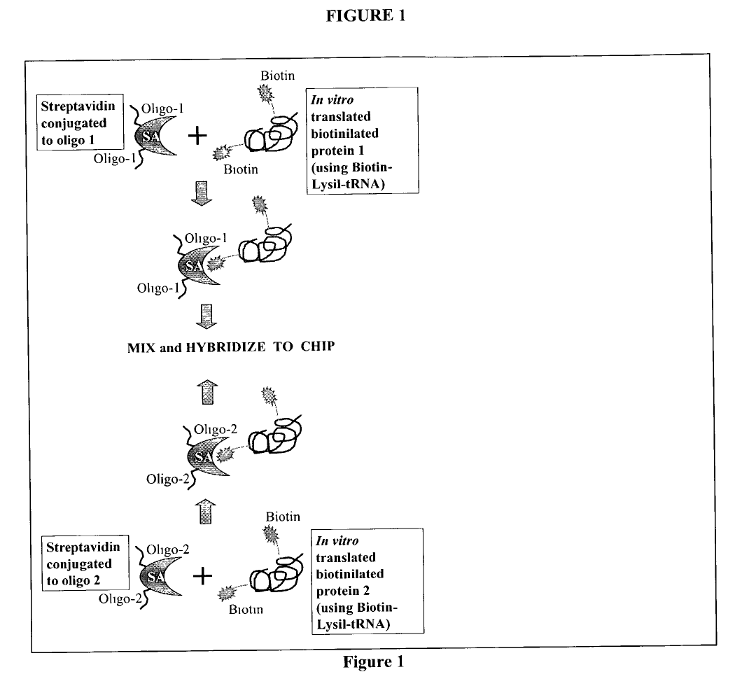

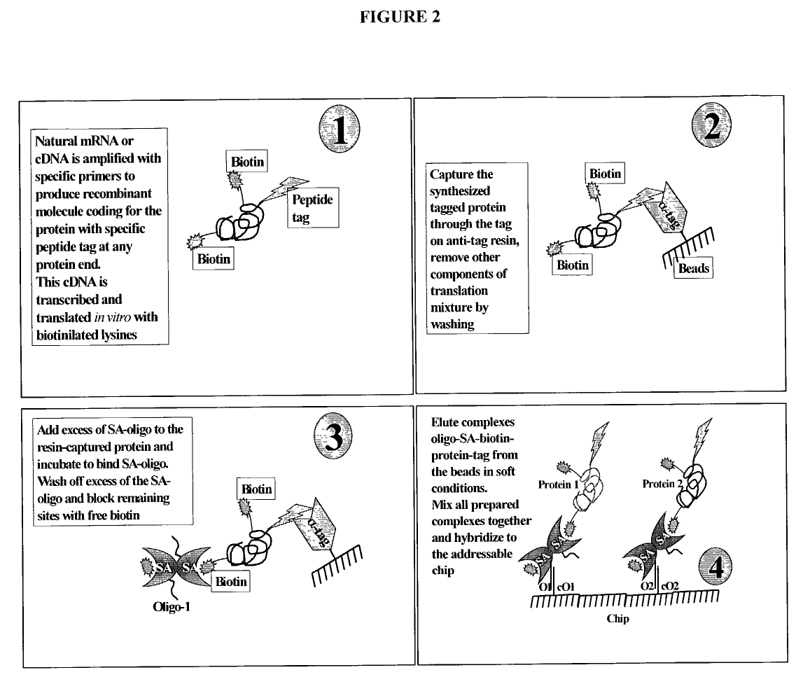

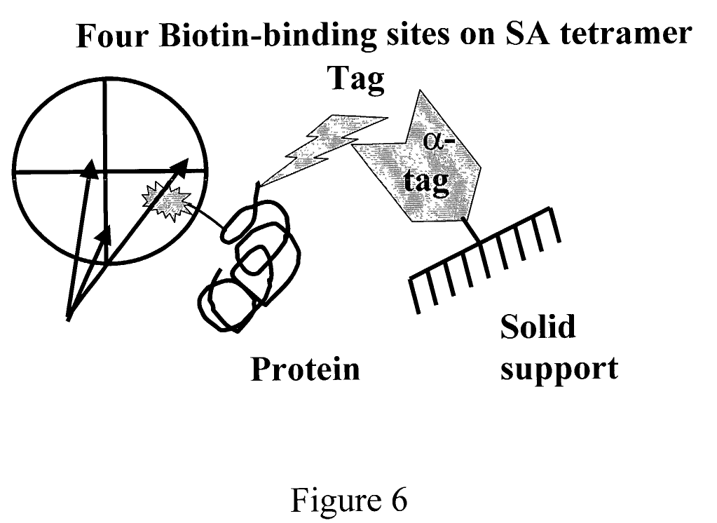

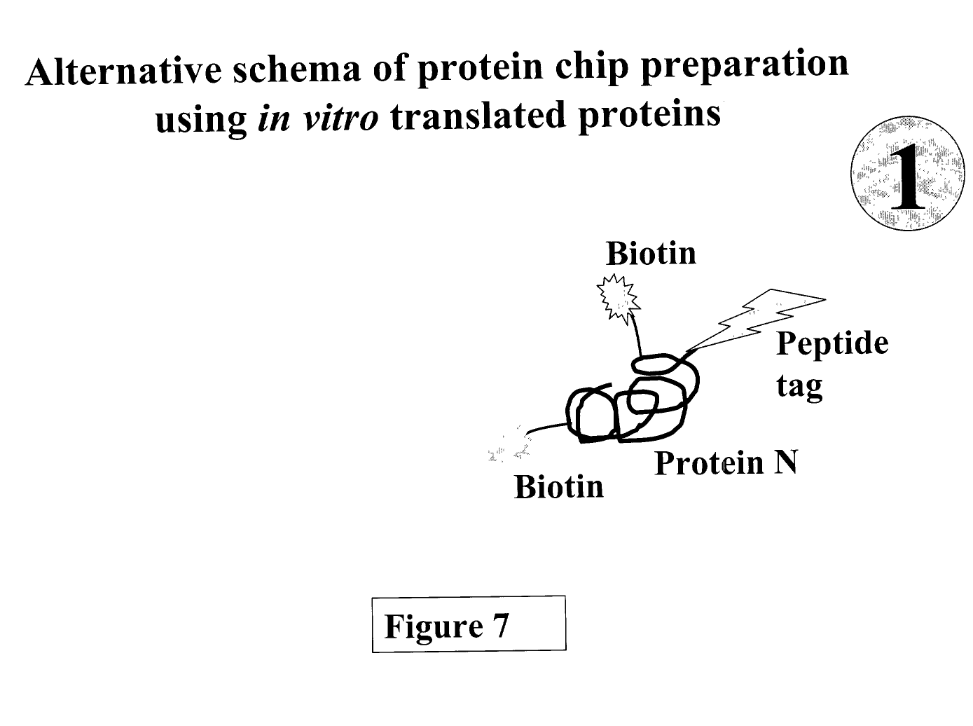

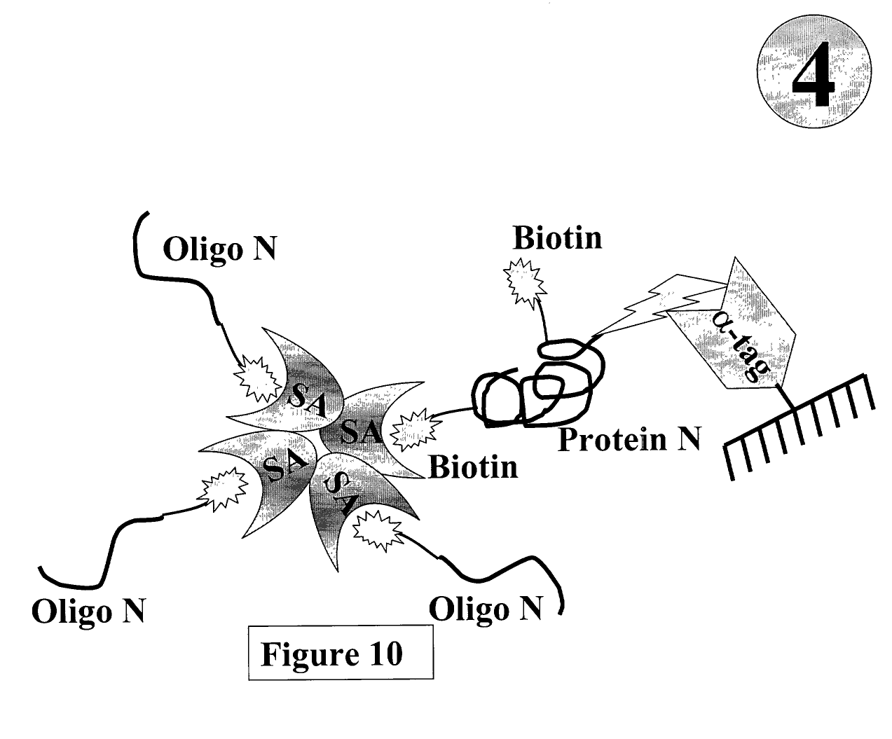

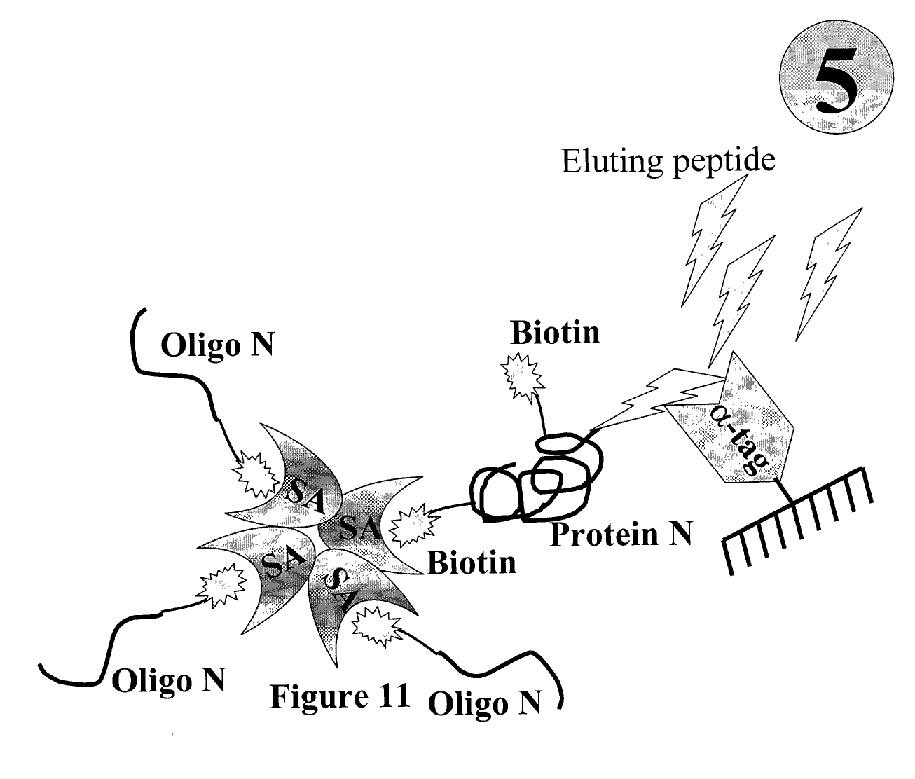

The present invention, in a preferred embodiment, can use stereo-chemical properties of linker molecules (e.g., streptavidin) to serve a dual purpose of capturing a synthetic protein (non-specifically biotin-labeled) and capturing specific oligonucleotide tags for self-assembly of the entire protein complex moiety onto a tag array microarray device (FIG. 6). The linker molecules provide label (e.g., biotinylated lisyl-tRNA in FIG. 1) and are preferably removed before capturing the synthetic protein by oligonucleotide-tagged streptavidin (SA) to avoid competition. At least one oligo-SA molecule is bound to each molecule of synthetic protein to obtain maximum amount of this protein on the microarray device after hybridization of the oligo-SA::biotinylated protein complexes. The yield of synthesis for different proteins may vary significantly. Therefore, to make the process more robust and not depending on the synthesis yield, excess oligo-SA can be added to the biotinylated synthetic protein for efficient binding, and afterwards free oligo-SA can be removed (FIG. 2). Moreover, streptavidin is a tetrameric protein with four sites to bind biotin. Therefore, all free sites, which are not occupied by biotinylated synthetic protein, should be blocked to preclude interaction of oligo-SA with another biotinylated synthetic protein after they are mixed for hybridization on the microarray device. To address these three issues, FIGS. 7-13 illustrated a preferred approach.

The examples below illustrate the ability to make protein microarrays according to the inventive process wherein the yields of protein are significantly greater because the in vitro translation step has significant efficiency because the mRNA molecule generated are used repeatedly to make many copies of a protein. This is in contrast to puromycin-based techniques wherein each mRNA molecule formed can only be used to make one protein molecule due to cross-linking issues.

In detail, different cDNAs corresponding to different proteins are prepared by polymerase chain reaction (PCR) using specific oligonucleotide primers and commercially available cDNA libraries. Elements such as T7 RNA polymerase promoter, ribosome binding sequences, specific peptide epitopes and the like are added through synthesis of the specific PCR primers. These synthetic cDNAs, each in separate tube or well of a microtiter plate, are used for in vitro transcription and translation of the corresponding proteins.

First binding moieties, such as biotin, are introduced randomly in the synthetic proteins during in vitro translation using biotinylated lysil-tRNA. Other first binding moieties are also used and incorporated into protein chains. These synthetic proteins are bound to the tagged capturing molecules of second binding moieties. When biotin is used as the first binding moiety, the second binding moiety is streptavidin or avidin. The tag array is an oligonucleotide sequence of from about 10 to about 100 bases long that is unique for each synthetic protein and is conjugated to the second binding moiety. The complexes comprising a synthetic protein plus oligonucleotide-tagged streptavidin bound to a first binding moiety on the synthetic protein are mixed together. The mixture is added to a tag array microarray device containing complementary (to the unique oligonucleotide tag sequence) sequences at known locations such that each unique specific protein will localize to its specific complement oligonucleotide tag sequence at a known location on the microarray, thereby making a self-assembled protein microarray. After the self-assembly and appropriate washing step the microarray will be ready for use.

Methods for making oligonucleotide tag array microarrays are known and generally fall into three categories, spotting (including ink jet printing techniques either of the entire oligonucleotide sequence or by in situ oligonucleotide synthesis); photolithography using mask sets to synthesize oligonucleotides in situ or through a mirror-based laser light system with photo-cleavable blocking groups using standard phosphoramidite chemistry techniques; through electric field-based localization of fully-formed oligonucleotides through an electric charge; or through in situ synthesis electrochemistry to use electrodes to generate electrochemical reagents to deblock phosphoramidite-based nucleotides to allow for new base addition.

The present process for making protein microarrays provides significantly greater yields of protein because the in vitro translation step has significant efficiency due to the repeated use of the mRNA molecule to make many copies of a protein. This is in contrast to puromycin-based techniques wherein each mRNA molecule formed can only be used to make one protein molecule due to cross-linking issues.

With regard to FIG. 1, this shows a simplified scheme of a preferred embodiment of the inventive process. Several advantages are associated with this schema. First, the random orientation of the biotin moieties provides the random orientation of the proteins on the microarray device including the orientations that do not affect protein activity. Second, this schema is automated and uses recent advances in in vitro translation methods to build a protein mircoarray product having a plurality of different properly folded (for protein or enzymatic activity) proteins at known locations on a microarray device.

To synthesize proteins in cell-free system one can use synthetic mRNAs prepared by in vitro transcription of corresponding cDNAs using T7 RNA polymerase or other RNA polymerases, or a coupled transcription or translation procedure. Complementary DNA used in this procedure is prepared by PCR from the commercial cDNA libraries expressing desirable proteins. During PCR, T7 RNA-polymerase promoter sequence is introduced and some other short sequences (such as ribosome binding site, peptide epitope, and the like) using appropriately designed primers. A combined transcription/translation system is also possible.

In a preferred embodiment, the molecules providing label (biotinylated lysil-tRNA in FIG. 1) are removed before capturing synthetic protein by oligonucleotide-tagged streptavidin (SA) to avoid competition. At least one oligonucleotide-SA molecule should be bound to each molecule of synthetic protein to obtain maximum amount of this protein on the microarray device after hybridization of the oligonucleotide-SA::biotinilated protein complexes. The yield of synthesis for different proteins may vary significantly. Therefore, to make the process more robust and not depending on the synthesis yield, it is better to add excess of oligonucleotide-SA to the biotinylated synthetic protein for efficient binding and then remove access of the free oligo-SA. Moreover, SA is a tetrameric protein with four sites to bind biotin. Therefore, all free sites, which are not occupied by biotinylated synthetic protein, should be blocked to preclude interaction of oligonucleotide-SA with another biotinylated synthetic protein after they mixed for hybridization on the microarray device. To address these three issues, a preferred approach has the following 4 steps shown in FIGS. 2 and 7-13.

An additional peptide epitope is easy to add during PCR similarly to the addition of the T7 promoter using the appropriate primers. The addition of a short peptide (about 8 amino acid residues) in general should not affect the protein folding and activity. However, if this is the case, the opportunity to place it to N- or C-terminal end should help to solve this problem as well. Another advantage of using a short peptide epitope is a possibility to elute the oligonucleotide-SA::protein complexes in soft conditions using competition with free peptide tag. In the examples, commercially available FLAG epitope and anti-FLAG resin was used. Different peptide epitopes of similar length are designed and synthesized. Antibody against it is raised, purified and coupled to the resin.

Coupling of SA (streptavidin) molecules to tagging oligonucleotides is a non-controllable chemical reaction. As SA is a tetramer, it can bind to up to four biotin moieties. When synthetic protein is bound to a solid support through a peptide tag (FIG. 6) or any other means, SA tetramers can bind this protein through incorporated biotin moieties only through one biotin-binding site leaving three other sites available for interaction with biotin. The three remaining sites can be filled in using specific oligonucleotides coupled with biotin moieties providing specific tagging for the complex of synthetic protein with incorporated biotins bound to SA. Thus, this step eliminates the need for chemical coupling of SA with specific oligonucleotides and makes the inventive process available for automated techniques.

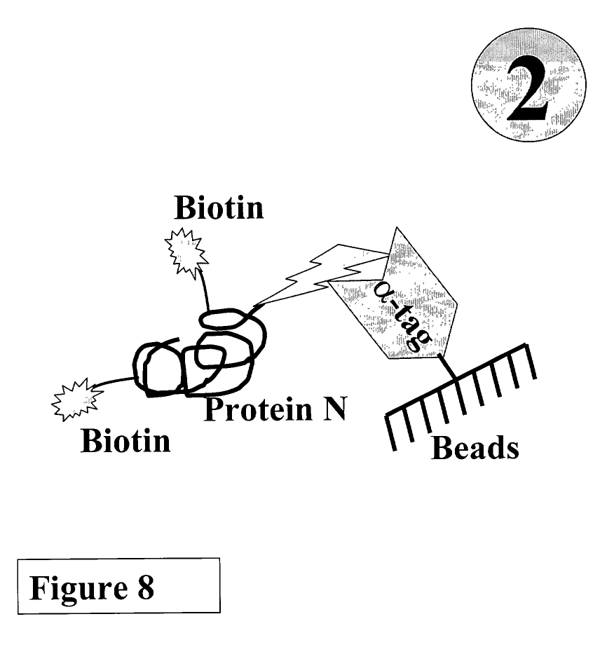

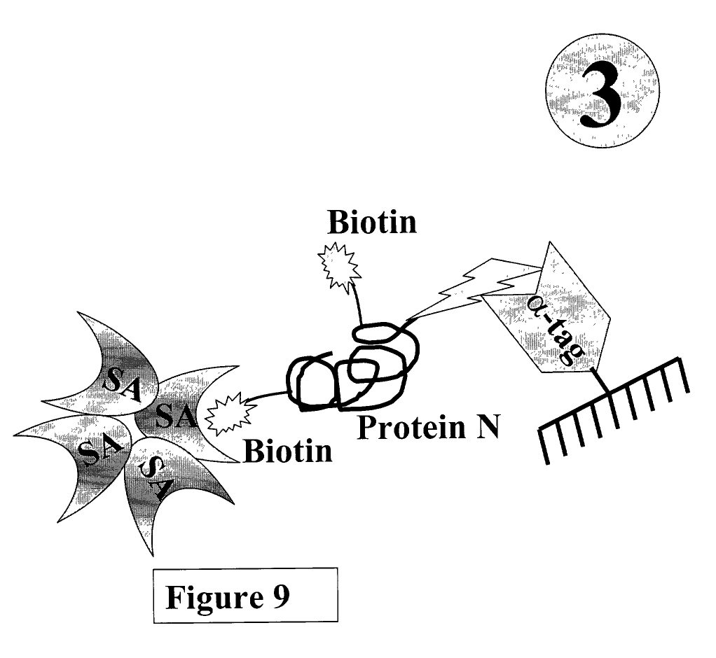

In a preferred embodiment, the synthetic protein is first bound to a solid support after synthesis. Those moieties providing a label (e.g., first binding moieties, biotinylated lysil-tRNA in FIG. 8) are removed by washing.

The following examples are given for the purpose of illustrating various embodiments of the invention and are not meant to limit the present invention in any fashion.

### EXAMPLE 1

Luciferase-FLAG Fusion Protein Preparation

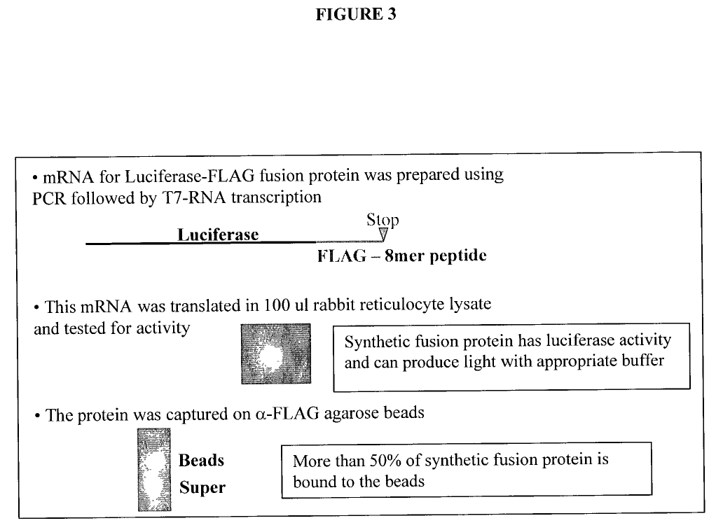

Luciferase control DNA (Promega) was used to prepare DNA coding for Luciferase-FLAG fusion protein by conventional methods of PCR with appropriate sequences. A commercially available FLAG epitope (e.g., a FLAG eight amino acid additional sequence (FIG. 3) was added for immobilizing the translated fusion protein. In general, a short peptide (e.g., about 8 amino acid residues) should not affect the tertiary protein folding and biological activity. However, if this is the case, the opportunity to place it to N- or C-terminal end should help to solve this problem as well. Additionally using a short peptide epitope provides a possibility of eluting the oligo-SA::protein::protein complexes in soft conditions using competition with free peptide tag. The final construct included T7 RNA-polymerase promoter, Kozak sequence for initiation of translation, Luciferase coding sequence fused to FLAG-coding sequence, and stop-codon. This DNA was transcribed and mRNA was purified using Promega's mRNA synthesis kit. Concentration of 1.65 mg/ml was obtained. This mRNA was used for translation in vitro using Promega's Flexi Rabbit Reticulocyte Lysate translation system (final volume 300 μl) and 6 μl of biotinylated lysine-tRNA (Transcend t-RNA, Promega).

### EXAMPLE 2

EGFP-FLAG Fusion Protein Preparation

pEGFP-N2 plasmid (Clontech) was used to amplify, by conventional PCR methods, the EGFP-coding DNA and to add FLAG-coding sequence in frame with the 3′-terminus of EGFP-coding sequence and T7 and Kozak sequences at the 5′-end of the DNA. This construct was used for coupled transcription/translation using Promega's TnT T7 PCR Quick transcription/translation system (total volume 300 μl) and 6 μl of Transcend biotinylated lysine-tRNA (Promega).

### EXAMPLE 3

Capture of the Synthetic Proteins on Solid-Phase for Preparation of Oligonucleotide-Tagged Complexes

Anti-FLAG M2 resin (Sigma) was used to capture each synthetic protein in separate tube after translation. 25 μl of resin was added and incubated with translation mixtures overnight at 4° C. Resin was washed 5 times in TBS buffer (10 mM Tris-HCl, pH 7.6, 150 mM NaCl). EGFP protein bound to the resin was visible (as green fluorescence) after this step under appropriate blue light.

FIG. 3 demonstrates the design, synthesis and immobilization on the resin of Luciferase-FLAG hybrid molecules. A commercial anti-FLAG antibody (α-FLAG) (immobilized onto beads) was able to capture the translated fusion protein. Moreover, the ability of the translated protein to properly fold (i.e., have proper tertiary structure) was demonstrated by the insert showing light production catalyzed by the translated fusion protein. This indicates that addition of the FLAG peptide to luciferase and incorporation of biotin moieties does not affect luciferase activity and that this fusion protein could be effectively captured on the anti-FLAG resin.

### EXAMPLE 4

Tagging Synthetic Proteins with Specific Oligonucleotides

100 μl of 0.1 mg/ml streptavidin (SA) in 10% BSA in PBS (Pierce) solution was added to each tube and incubated for several hours at 4° C. to bind to biotinylated synthetic proteins captured on the anti-FLAG resin. The resin was washed 3 times with TBS and excess of biotinylated oligonucleotides (5 μl of 100 μM concentration in 100 μl of 10% BSA in PBS) was added to each tube to bind to tag the synthetic protein-SA complexes. Each oligonucleotide was designed in a way that they did not cross-hybridize to each other, and to the other oligonucleotides synthesized on or spotted on a microarray device for capturing tagged protein complexes. Each synthetic protein was tagged with a specific oligonucleotide. The resin was washed 5 times in TBS. Tagged complexes were eluted using 50 μl of 0.3 mg/ml 3× FLAG peptide (Sigma) in 10% BSA-PBS solution. This scheme is illustrated in FIGS. 7-11.

### EXAMPLE 5

Self-Assembling of Tagged Protein Complexes on a Microarray Device

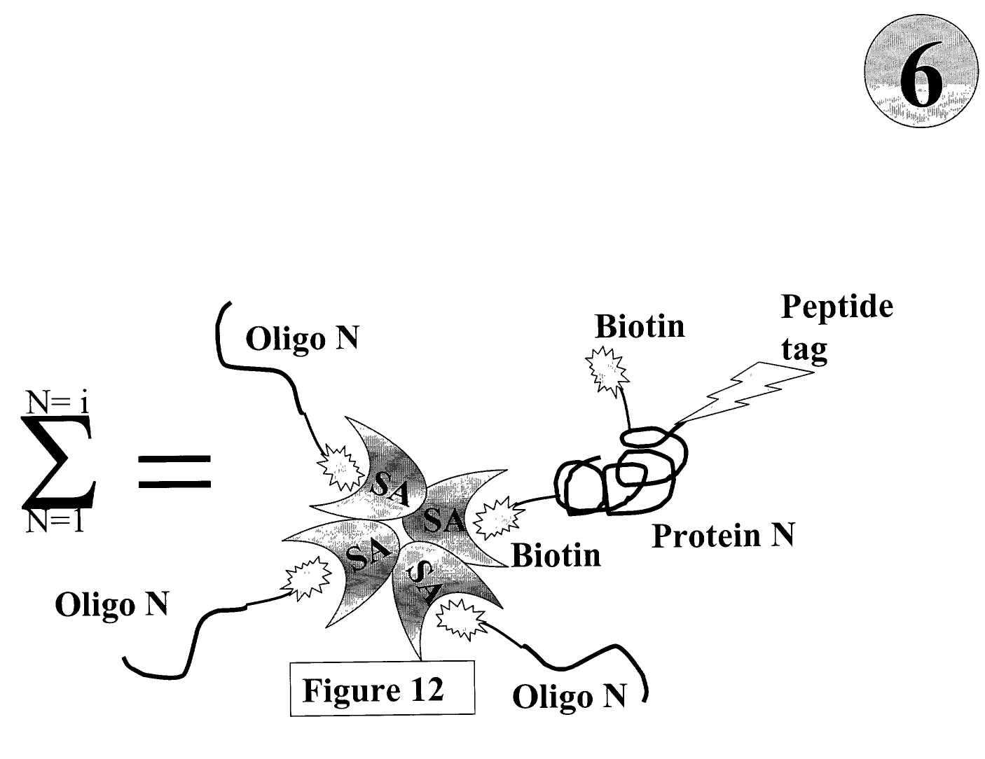

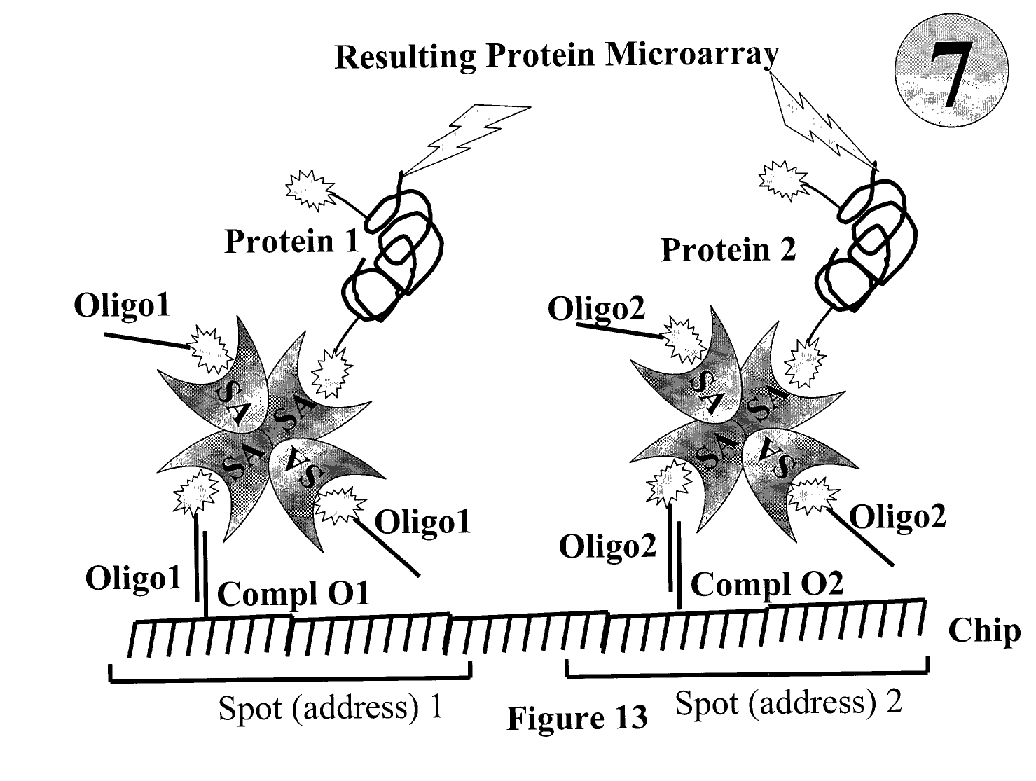

Tagged complexes were mixed together with NaCl. The concentration of the mixture was adjusted to 0.15-0.3 mM NaCl. This mixture was incubated with a microarray device containing ten different capturing oligonucleotides made by in situ electrochemistry (Combimatrix Corporation, Mukilteo, Wash.), two of which were complementary to the two biotinylated oligonucleotides used to tag synthetic proteins. Microarrays were or were not pre-incubated with blocking solution (10% BSA in PBS) to reduce non-specific binding. Incubation of tagged protein complexes was performed at 37-40° C. for 1-16 hours. Microarrays were washed 3 times in 2× TBS solution to remove non-bound complexes. After this washing step protein microarrays were considered prepared and ready for analysis of the bound proteins in different biochemical assays. This step is illustrated in FIG. 12.

### EXAMPLE 6

Detection of Specific Capture of the Synthetic Proteins

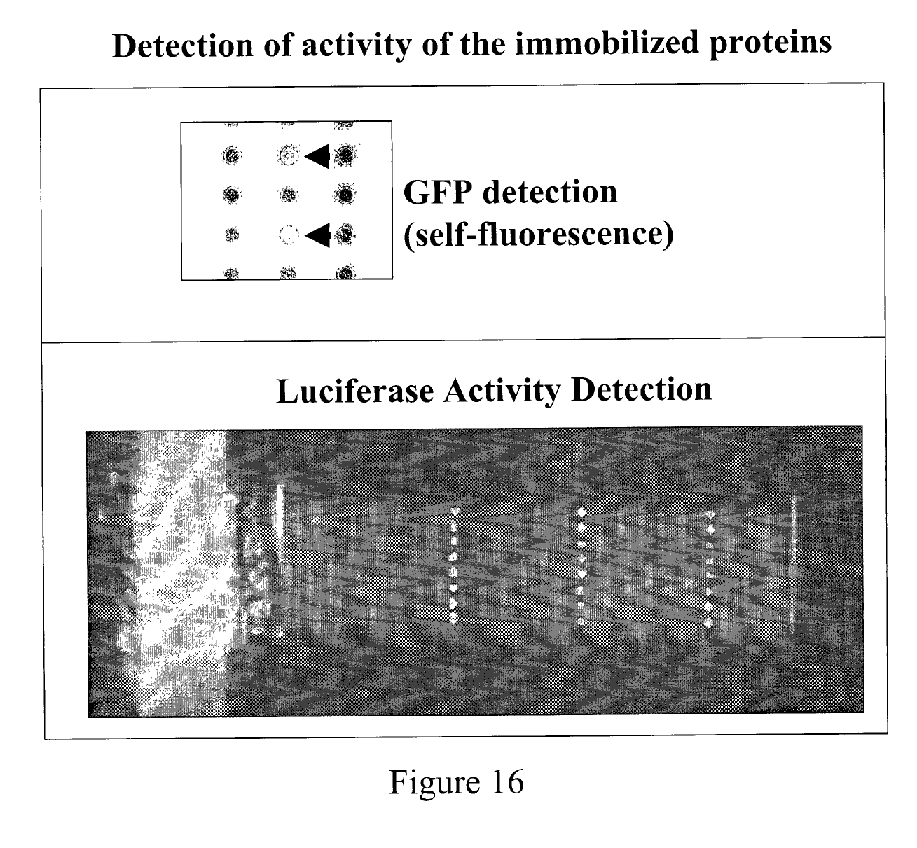

Detection by functional activity: EGFP was detected at specific locations designed for its placement by self-fluorescence under appropriate blue light. Luc was detected at specific locations designed for its placement by reaction with substrate (Luciferase assay reagent, Promega) and able to produce chemiluminescence, detectable by a CCD camera device. FIG. 16 shows detection of both proteins in a CCD camera image.

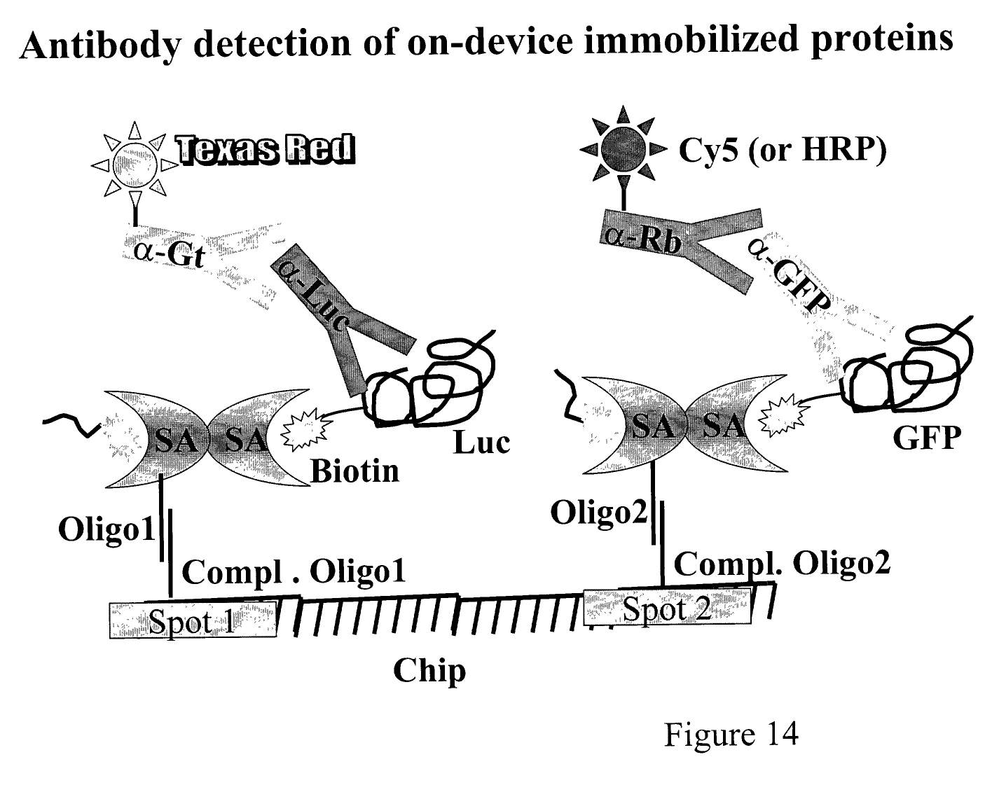

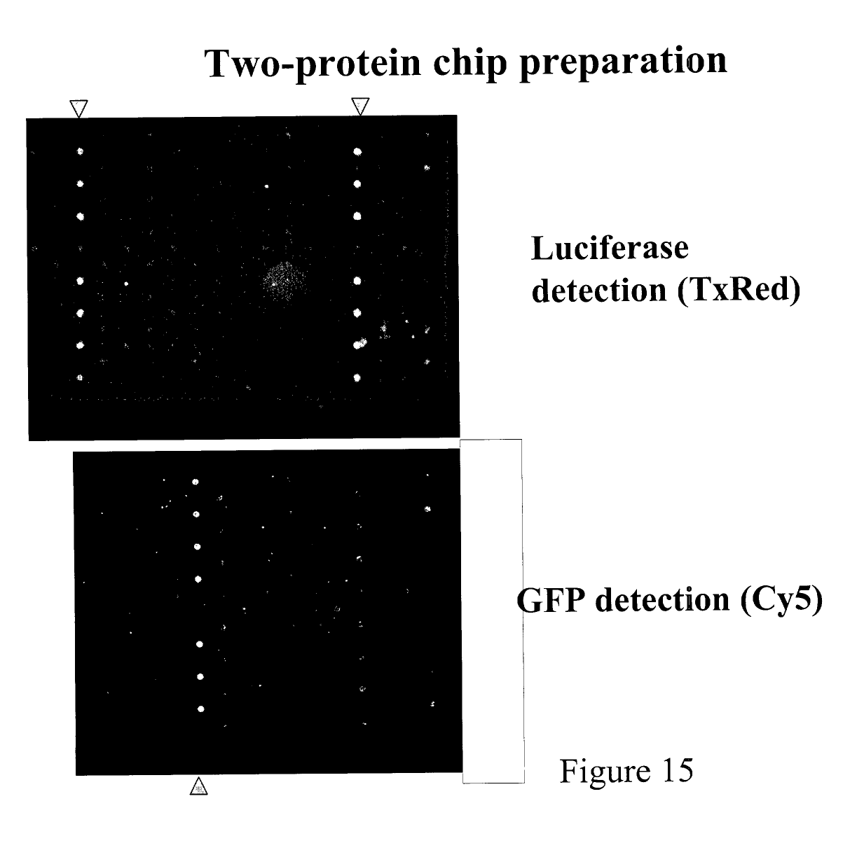

Detection by antibody reactivity: Proteins immobilized on the microarray were detected in a sandwich assay. Each protein microarray was incubated with anti-luciferase goat antibody (Promega) and anti-GFP rabbit antibody (Clontech) in 2× TBS, 10% BSA solution for 2 hours at room temperature. Microarrays were washed 4 times in 2× TBS and incubated with secondary antibodies labeled with different fluorescence labels (Jackson ImmunoResearch Laboratories). Mouse anti-Rabbit antibody was labeled with Cy-5® fluorescent dye, and mouse anti-goat antibody was labeled with Texas Red® fluorescent dye. After 1-hour incubation the microarrays were washed in 2× TBS. Signals from antibodies were visualized at appropriate wavelength (FIG. 14 for images of microarray device).

### EXAMPLE 7

Preparation of SA-Oligo1 Conjugates and Testing of Biotin-Binding and Specific Addressing on a Microarray

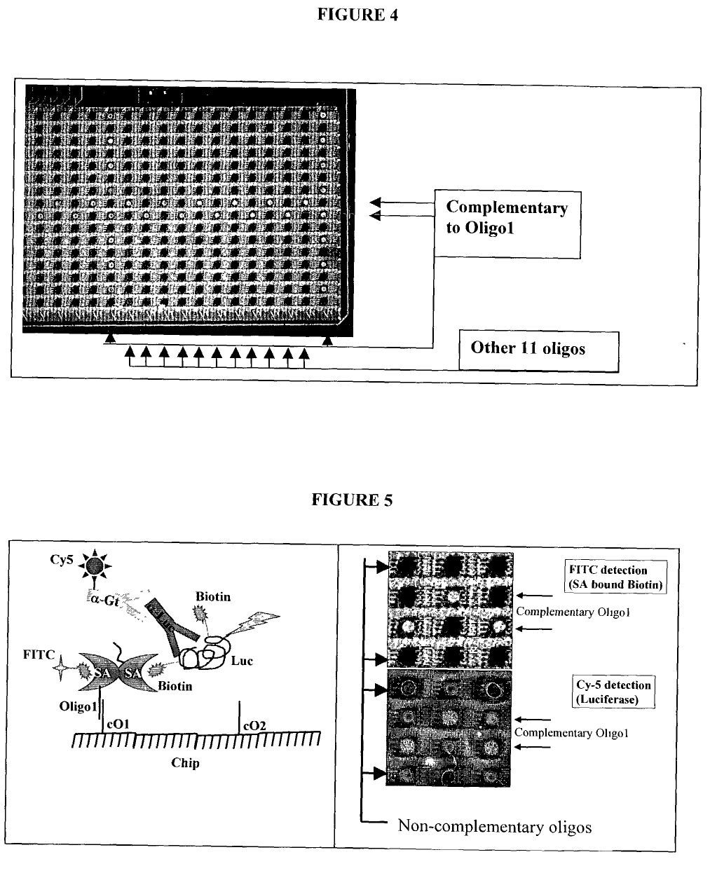

Streptavidin (SA) was treated with Traut's reagent (Pierce) to introduce SH groups to the protein. Oligonucleotide 1 (oligo1) was synthesized with an amino-group at the 3′ end. The modified protein was coupled to this oligo1 using heterobifunctional reagent maleimidobenzoyl-N-hydrosuccinimide ester (Pierce). This SA-oligo1 conjugate was incubated with biotin-labeled with fluorescein-isotiocyanate (FITC) and then hybridized to the microarray with 12 different oligonucleotide sequences synthesized in specific order (FIG. 4). Presence of biotin-FITC was detected using a fluorescence microscope and a CCD camera. The data indicate that SA-oligo conjugates preserved their ability to bind biotin and could be specifically addressed to the desired position on the microarray.

This approach may be improved by introduction of a cysteine amino acid residue into SA. By doing so, the step required for modification of SA with Traut's reagent will be eliminated, thereby make it more uniform since one SH-group is always available on the protein for the oligonucleotide conjugation.

### EXAMPLE 8

Preparation of the Protein Microarray Prototype Device Using Single Protein: Luciferase-FLAG

Resin-bound luciferase-FLAG was incubated with SA-oligo1. Resin was then washed and streptavidin blocked by incubation with biotin-FITC. The luciferase-FLAG::SA-oligo1 complexes were eluted from the resin by FLAG peptide and hybridized to the 12-oligos microarray as described above. Presence of luciferase on the microarray was demonstrated by incubation of the microarray with goat anti-luciferase antibody followed by incubation with anti-goat IgG antibody labeled with Cy5® fluorescent dye. The schema of this experiment and the results are shown in FIG. 5. It is demonstrated that a protein of 60 kDa could be synthesized in a cell-free system and successfully immobilized on an oligonucleotide tag array microarray device at desired address. After immobilization, this protein preserved its ability to interact with other molecules (i.e., biological activity) demonstrated by its reactivity with antibody (a molecule of 150 kDa).

### EXAMPLE 9

Design of 10 Different Oligonucleotide Tags to Be Used for Ten-Proteins Microarray Device

To build a protein microarray device with 10 different proteins immobilized thereon, 10 different oligonucleotide sequences were designed with minimum homology to each other and similar melting temperatures using a proprietary oligonucleotide probe software program. However, other oligonucleotide probe design software programs are commercially available (e.g., Clontech) and can be used to design tag array microarrays. The oligonucleotide sequences were hybridized in different combinations to the microarray containing corresponding complementary oligonucleotide sequences. The results revealed no cross-hybridization among the oligonucleotide sequences.

### EXAMPLE 10

Ten-Protein Microarrays Preparation

Ten proteins of known sequence were chosen to be prepared and placed on the microarray device, including: folding marker (green fluorescence protein); enzymes (luciferase, secreted form of human alkaline phosphatase); targets for modification (histone H1, signaling protein Elk1 from Ras signaling pathway); protein-protein interaction (LDL receptor family, cytoplasmic tail of megalin, LDL-receptor, LRP, VLDL-receptor, and ApoE-receptor).

To prepare the ten-protein microarray, similar procedures are used as described above. Briefly, oligonucleotide primers are first designed and prepared to amplify proteins of interest. Primers contain T7 RNA polymerase promoter sequence, Kozak sequence for enhanced ribosome binding, and FLAG epitope. Next, individual streptavidin preparations are labeled with ten different tag oligonucleotides. Corresponding cDNA's are amplified using appropriate vectors with cloned genes or human PCR-ready kidney or brain cDNA libraries (Clontech), and further transcribed and translated in cell-free lysate (rabbit or wheat germ, Promega) using biotin-labeled Lys-tRNA (Promega). However, any cell free translation system can be used. The scale of transcription/translation is monitored, so that the amount of proteins is satisfactory for detection on microarrays with corresponding antibodies, thereby obtaining positive detection in functional tests. Synthesized proteins are then tested by electrophoresis and Western blotting.

Next, each synthesized protein is captured on anti-FLAG agarose beads (Sigma). The beads are washed off all components of the translation mixture. Excess of different SA-oligonucleotide sequence conjugates is added to each batch of synthetic protein bound to anti-FLAG agarose, and proceeded for incubation. Excess of SA-oligonucleotides is washed off. Available biotin sites on SA are blocked by incubation with excess of biotin. Oligo-SA::synthetic protein complexes are further eluted by free FLAG peptide.

After the formation of the oligo-SA::synthetic protein complexes, all ten complexes are mixed together and incubated with appropriate microarrays having complementary tag oligonucleotide sequences at known locations (e.g., Combimatrix Corporation, Mukilteo, Wash.). Microarrays are then washed off and proceeded for testing for the presence of synthetic proteins using fluorescent dye labeled antibodies, fluorescence microscope and/or digital camera.

The above mentioned approach is optimized to obtain maximal possible amounts of the microarray-immobilized proteins by adjusting scale and salt composition of transcription/translation reactions, protein concentrations, time of hybridization, and/or by variation of 5′-untranslated region, which is important for efficient translation. Other peptide tags can also be designed. Monoclonal or polyclonal antibody might be prepared against these tags and used instead of FLAG peptide.

### EXAMPLE 11

Analyze the Ten-Protein Microarray Device in Different Functional Experiments

The manufactured protein microarrays are tested for correct folding, protein-protein interactions and enzymatic and target activity of the immobilized proteins. Specifically, to test correct folding of the synthetic proteins, green fluorescent protein (GFP) is used for on-device immobilization. Its fluorescence will indicate the correct folding. Also, functional activity of other immobilized proteins, such as luciferase and alkaline phosphatase, indicate correct folding and the presence of proper biological activity.

To test on-device enzymatic activity, two enzymes have been chosen: luciferase and alkaline phosphatase. For both proteins there are commercially available chemiluminescent substrates that can be detected by CCD camera.

Target activity of the proteins immobilized on the microarrays is studied using protein kinases. Synthetic proteins containing specific phosphorylation sites (H1 histone and Elk1 protein kinase) are prepared and immobilized on the microarray. These proteins are tested for their ability to undergo phosphorylation by the appropriate protein kinase (PKA and Erk1).

Protein-protein interactions are tested in experiments exploiting previously characterized interacting pairs of proteins. Members of the low-density lipoprotein (LDL) receptor family (e.g., megalin) are tested for the interaction of their cytoplasmic tails immobilized on the microarray with Dab2 adaptor protein.

I claim:

1. A process for producing a self-assembled protein microarray having a plurality of proteins in discrete locations, comprising the steps of:
(a) preparing a plurality of cDNA's, each encoding a different protein, wherein each cDNA comprises a promoter region and a coding region and segregating each cDNA into separate chambers;
(b) transcribing each cDNA into a mRNA, wherein the mRNA will form a protein encoded by the coding region of the cDNA;
(c) translating each mRNA in a cell-free translation system to synthesize a plurality of synthetic proteins, wherein each synthetic protein includes a first binding moiety incorporated therein, and whereby each mRNA molecule can be used to translate a plurality of synthetic proteins to increase yield;
(d) attaching a second binding moiety that specifically binds to the first binding moiety, wherein the second binding moiety further comprises an oligonucleotide tag sequence to form a oligonucleotide-addressed synthetic protein; and
(e) localizing the oligonucleotide-addressed synthetic proteins onto an oligonucleotide tag mircoarray device, wherein the oligonucleotide tag mircoarray device comprises a plurality of oligonucleotide sequences at known locations, wherein then oligonucleotide sequences are designed to be complementary to an oligonucleotide tag sequence on the second binding moiety, whereby each oligonucleotide-addressed protein localizes to its predefined complementary region on the oligonucleotide tag mircoarray device through nucleic acid hybridization.

2. The process for producing a self-assembled protein microarray having a plurality of proteins in discrete locations of claim 1 wherein the cDNA's are prepared through PCR (polymerase chain reaction) techniques.

3. The process for producing a self-assembled protein microarray having a plurality of proteins in discrete locations of claim 1 wherein the cDNAs further comprises a tag region that codes on expression for a protein tag, wherein the protein tag sequence is used to affinity bind the synthetic protein in order to wash out unbound first binding moiety.

4. The process for producing a self-assembled protein microarray having a plurality of proteins in discrete locations of claim 1 wherein the translating step (c) further comprises adding cell or liver microsomes in order to provide for eukaryotic cell glycosylation of the synthetic protein at N-linked or O-linked glycosylation sites.

5. The process for producing a self-assembled protein microarray having a plurality of proteins in discrete locations of claim 1 wherein the first binding moiety is biotin or a biotin derivative thereof, and the second binding moiety is streptavidin or a streptavidin derivative thereof, or the first binding moiety is an antigen and the second binding moiety is an antibody of fragment thereof that binds to the first moiety antigen.

6. The process for producing a self-assembled protein microarray having a plurality of proteins in discrete locations of claim 5 wherein the first binding moiety is a biotin moiety that is linked to the synthetic peptide through Lys residues.

7. The process for producing a self-assembled protein microarray having a plurality of proteins in discrete locations of claim 1 wherein the oligonucleotide tag sequence attached to the second binding moiety is from about 12 to about 100 nucleotides in length wherein at least 12 nucleotides are exactly complimentary to their corresponding tag array oligonucleotide sequence on the mircoarray device.

8. A protein microarray having a plurality of proteins in discrete locations, wherein the protein mircoarray is produced by a process comprising the steps of:
(a) preparing a plurality of cDNA's, each encoding a different protein, wherein each cDNA comprises a promoter region and a coding region;
(b) transcribing each cDNA into a mRNA, wherein the mRNA will form a protein encoded by the coding region of the cDNA;
(c) translating each mRNA in a cell-free translation system to synthesize a plurality of different proteins, wherein each synthetic protein includes a first binding moiety incorporated therein, and whereby each mRNA molecule can be used to translate a plurality of synthetic proteins;
(d) attaching a second binding moiety that specifically binds to the first binding moiety, wherein the second binding moiety further comprises an oligonucleotide tag sequence to form a oligonucleotide-addressed synthetic protein; and
(e) localizing the oligonucleotide-addressed synthetic protein onto an oligonucleotide tag mircoarray device, wherein the oligonucleotide tag mircoarray device comprises a plurality of oligonucleotide sequences at known locations, wherein then oligonucleotide sequences are designed to be complementary to an oligonucleotide tag sequence on the second binding moiety, and whereby each oligonucleotide-addressed protein localizes to its predefined complementary region on the oligonucleotide tag mircoarray device through nucleic acid hybridization.

9. The protein microarray having a plurality of proteins in discrete locations of claim 8 wherein the cDNA's are prepared through PCR (polymerase chain reaction) techniques.

10. The protein microarray having a plurality of proteins in discrete locations of claim 8 wherein the cDNAs further comprises a tag region that codes on expression for a protein tag, wherein the protein tag sequence is used to affinity bind the synthetic protein in order to wash out unbound first binding moiety.

11. The protein microarray having a plurality of proteins in discrete locations of claim 8 wherein the translating step (c) further comprises adding cell or liver microsomes in order to provide for eukaryotic cell glycosylation of the synthetic protein at N-linked or O-linked glycosylation sites.

12. The protein microarray having a plurality of proteins in discrete locations of claim 8 wherein the first binding moiety is biotin or a biotin derivative thereof, and the second binding moiety is streptavidin or a streptavidin derivative thereof, or the first binding moiety is an antigenic epitope and the second binding moiety is an antibody or fragment thereof that binds to the first moiety antigen.

13. The protein microarray having a plurality of proteins in discrete locations of claim 12 wherein the first binding moiety is a biotin moiety that is linked to the synthetic peptide through Lys residues.

14. The protein microarray having a plurality of proteins in discrete locations of claim 8 wherein the oligonucleotide tag sequence attached to the second binding moiety is from about 12 to about 100 nucleotides in length wherein at least 12 nucleotides are exactly complimentary to their corresponding tag array oligonucleotide sequence on the mircoarray device.

15. A process for producing a self-assembled protein microarray having a plurality of proteins in discrete locations, comprising the steps of:
(a) preparing a plurality of cDNA's, each encoding a different protein, wherein each cDNA comprises a promoter region, a coding region and a tag region in frame to the coding region and segregating each cDNA into separate chambers;
(b) transcribing each cDNA into a mRNA, wherein the mRNA will form a protein encoded by the coding region of the cDNA and having a peptide tag sequence, wherein each peptide tag sequence is the same across the plurality of proteins;
(c) translating each mRNA in a cell-free translation system to synthesize a plurality of different proteins, each containing the same peptide tag sequence, to form the synthetic protein;
(d) incorporating in each translated protein a first binding moiety;
(e) removing the free first binding moiety by binding the synthetic protein through an affinity third binding moiety that binds to the peptide tag sequence and is attached to a solid phase, and washing to remove free or unbound first binding moiety;
(f) attaching a second binding moiety that specifically binds to the first binding moiety, wherein the second binding moiety further comprises an oligonucleotide tag sequence to form a oligonucleotide-addressed synthetic protein; and
(g) localizing the oligonucleotide-addressed protein onto an oligonucleotide tag mircoarray device, whereby each oligonucleotide-addressed protein localizes to its predefined complementary region on the oligonucleotide tag mircoarray device through nucleic acid hybridization.

16. The protein microarray having a plurality of proteins in discrete locations of claim 15 wherein the oligonucleotide-addressed synthetic proteins are first eluted before being localized onto the oligonucleotide tag microarray device.

17. The process for producing a self-assembled protein microarray having a plurality of proteins in discrete locations of claim 15 wherein the cDNA's are prepared through PCR (polymerase chain reaction) techniques.

18. The process for producing a self-assembled protein microarray having a plurality of proteins in discrete locations of claim 15 wherein the translating step (c) further comprises adding cell or liver microsomes in order to provide for eukaryotic cell glycosylation of the synthetic protein at N-linked or O-linked glycosylation sites.

19. The process for producing a self-assembled protein microarray having a plurality of proteins in discrete locations of claim 15 wherein the first binding moiety is biotin or a biotin derivative thereof, and the second binding moiety is streptavidin or a streptavidin derivative thereof, or the first binding moiety is an antigenic epitope and the second binding moiety is an antibody or fragment thereof that binds to the first moiety antigen.

20. The process for producing a self-assembled protein microarray having a plurality of proteins in discrete locations of claim 19 wherein the first binding moiety is a biotin moiety that is linked to the synthetic peptide through Lys residues.

21. The process for producing a self-assembled protein microarray having a plurality of proteins in discrete locations of claim 15 wherein the oligonucleotide tag sequence attached to the second binding moiety is from about 12 to about 100 nucleotides in length wherein at least 12 nucleotides are exactly complimentary to their corresponding tag array oligonucleotide sequence on the mircoarray device.

22. A protein microarray having a plurality of proteins in discrete locations, wherein the protein mircoarray is produced by a process comprising the steps of:
(a) preparing a plurality of cDNA's, each encoding a different protein, wherein each cDNA comprises a promoter region, a coding region and a tag region in frame to the coding region and segregating each cDNA into separate chambers;
(b) transcribing each cDNA into a mRNA, wherein the mRNA will form a protein encoded by the coding region of the cDNA and having a peptide tag sequence, wherein each peptide tag sequence is the same across the plurality of proteins;
(c) translating each mRNA in a cell-free translation system to synthesize a plurality of different proteins, each containing the same peptide tag sequence, to form the synthetic protein;
(d) incorporating in each translated protein a first binding moiety;
(e) removing the free first binding moiety by binding the synthetic protein through an affinity third binding moiety that binds to the peptide tag sequence and is attached to a solid phase, and washing to remove free or unbound first binding moiety;
(f) attaching a second binding moiety that specifically binds to the first binding moiety, wherein the second binding moiety further comprises an oligonucleotide tag sequence to form a oligonucleotide-addressed synthetic protein; and
(g) localizing the oligonucleotide-addressed protein onto an oligonucleotide tag mircoarray device, whereby each oligonucleotide-addressed protein localizes to its predefined complementary region on the oligonucleotide tag mircoarray device through nucleic acid hybridization.

23. The protein microarray having a plurality of proteins in discrete locations of claim 22 wherein the cDNA's are prepared through PCR (polymerase chain reaction) techniques.

24. The protein microarray having a plurality of proteins in discrete locations of claim 22 wherein the translating step (c) further comprises adding cell or liver microsomes in order to provide for eukaryotic cell glycosylation of the synthetic protein at N-linked or O-linked glycosylation sites.

25. The protein microarray having a plurality of proteins in discrete locations of claim 22 wherein the first binding moiety is biotin or a biotin derivative thereof, and the second binding moiety is streptavidin or a streptavidin derivative thereof, or the first binding moiety is an antigenic epitope and the second binding moiety is an antibody or fragment thereof that binds to the first moiety antigen.

26. The protein microarray having a plurality of proteins in discrete locations of claim 25 wherein the first binding moiety is a biotin moiety that is linked to the synthetic peptide through Lys residues.

27. The protein microarray having a plurality of proteins in discrete locations of claim 22 wherein the oligonucleotide tag sequence attached to the second binding moiety is from about 12 to about 100 nucleotides in length wherein at least 12 nucleotides are exactly complimentary to their corresponding tag array oligonucleotide sequence on the mircoarray device.

28. A process for producing a self-assembled protein microarray having a plurality of proteins in discrete locations, comprising: (a) preparing a plurality of cDNA's in separate containers, wherein each cDNA encodes a different protein; (b) amplifying each cDNA with specific primers to produce a plurality of synthetic proteins, wherein each synthetic protein contains a peptide tag at either terminus; (c) incorporating in each synthetic protein a first binding moiety by in vitro translation; (d) capturing each synthetic protein on a solid phase using an antibody directed against the peptide tag; (e) adding a second binding moiety to each synthetic protein in each container, wherein the second binding moiety is multivalent and binds specifically to the first binding moiety, (f) adding a plurality of oligonucleotides labeled with the first binding moiety to bind to the second binding moiety, thereby forming an oligonucleotide-tagged protein complex, wherein the different tag oligonucleotides are designed in a way that they do not cross-hybridize to each other, (g) eluting each oligonucleotide-tagged protein complex from the solid phase; and (h) mixing the oligonucleotide-tagged protein complexes from separate containers and incubating the mixture with an oligonucleotide tag microarray device, wherein each oligonucleotide-tagged protein complex localizes to its predefined complementary region on the oligonucleotide tag microarray device, thereby forming a self-assembled protein microarray having a plurality of proteins in discrete locations.

29. The protein microarray having a plurality of proteins in discrete locations of claim 28 wherein the cDNA's are prepared through PCR techniques.

30. The protein microarray having a plurality of proteins in discrete locations of claim 28 wherein the first binding moiety is an antigenic epitope or a fragment thereof, and the second binding moiety is an antibody or a fragment thereof that binds to the first binding moiety.

31. The protein microarray having a plurality of proteins in discrete locations of claim 28 wherein the first binding moiety is biotin or a biotin derivative thereof, and the second binding moiety is streptavidin or a strepavidin derivative thereof.

32. The protein microarray having a plurality of proteins in discrete locations of claim 28 wherein the biotin moiety is linked to the synthetic protein through Lys residues.

33. The protein microarray having a plurality of proteins in discrete locations of claim 28 wherein the tag oligonucleotide conjugated to the second binding moiety is from about 10 to about 100 nucleotides in length, wherein at least 12 nucleotides are exactly complimentary to their corresponding oligonucleotide sequence on the oligonucleotide tag microarray device.
